(19) United States
(12) Patent Application Publication
(10) Pub. No.: US 2008/0269692 A1
(43) Pub. Date: Oct. 30, 2008

(54) Apparatus for Injecting a Pharmaceutical

(76) Inventors: Adrian Benton James, Palo Alto, CA (US); Robert Ian Lister, Sheffield (GB); Peter Scott MacDonald, Palo Alto, CA (US)

Correspondence Address: ELI LILLY & COMPANY, PATENT DIVISION, P.O. BOX 6288, INDIANAPOLIS, IN, 46206-6288, US

(21) Appl. No.: 12/089,620

(22) PCT Filed: Oct. 10, 2006
(86) PCT No.: PCT/US06/39390

§ 371 Date: Apr. 9, 2008

Related U.S. Application Data
(60) Provisional application No. 60/725,236, filed on Oct. 11, 2005.

(51) Int. Cl. A61M 5/32; A61M 5/31

(52) U.S. Cl. 604/198; 604/218

(57) ABSTRACT

A pharmaceutical delivery apparatus including a housing (22), a syringe assembly, and a needle (44) cap. The syringe assembly is plungeable relative to the housing from a first position, at which its needle tip is disposed within the housing, to a second position, at which its needle tip projects from the housing beyond the proximal end for insertion into an injection site. A base (46) of the needle cap is exposed at the housing proximal end to be manually grippable for cap removal. A needle cap stem is upstanding from the base and sized and configured to insert through an opening is the housing proximal end to cover the needle tip when the syringe assembly is disposed in the first position. The needle cap base further includes a plurality of distally projecting cams (54, 258) located radially outward of the stem. The cams are fittable within slots (40, 240) in the housing proximal end when the cap is fully mounted to the apparatus.

### BACKGROUND OF THE INVENTION

The present invention pertains to pharmaceutical delivery devices, and, in particular, to a manually powered delivery device for injecting a pharmaceutical.

Patients suffering from a number of different diseases frequently must inject themselves with pharmaceuticals. As some patients find it difficult to insert a standard syringe needle into one's skin and then operate the syringe to inject the pharmaceutical, a variety of devices have been proposed to facilitate the injection process.

One type of device automatically inserts a needle and then automatically injects a dose of medication through that inserted needle. While useful, these devices may be expensive to provide due to their complexity, and further may be undesirable to users who want more control over the injection process.

A wide assortment of injection pens are also available, which pens make manual injections easier for some people. However, most such pens, which may be suited for variable dose injections, are unnecessarily complicated if needed for only a single use.

Thus, it would be desirable to provide a device that can overcome one or more of these and other shortcomings of the prior art.

### BRIEF SUMMARY OF THE INVENTION

In one form thereof, the present invention provides a pharmaceutical delivery apparatus comprising a housing extending between a distal end and a proximal end, a syringe assembly including a needle having a proximal tip, the assembly plungeable relative to the housing from a first position, at which the needle tip is disposed within the housing, to a second position, at which the needle tip projects from the housing beyond the proximal end for insertion into an injection site, and a needle cap including a base and a stem. The base is exposed at the housing proximal end to be manually grippable for cap removal. The stem is upstanding from the base and sized and configured to insert through an opening is the housing proximal end to cover the needle tip when the syringe assembly is disposed in the first position. The needle cap base further includes a plurality of distally projecting cams located radially outward of the stem. The cams are fittable within slots in the housing proximal end when the cap is fully mounted to the apparatus. The cams and slots are complementarily sized and configured whereby twisting of the fully mounted cap relative to the housing causes the cams to cammably slide along surfaces of the housing proximal end to shift the cap proximally relative to the housing.

One advantage of the present invention is that a single use medication delivery device may be provided which is intuitive and easy to operate.

Another advantage of the present invention is that a medication delivery device may be provided which stages use of the device.

Yet another advantage of the present invention is that a medication delivery device may be provided which allows its needle to be manually retracted for locking in a simple manner once the device has been used for administration.

Still another advantage of the present invention is that a medication delivery device may be provided with an easy to remove cap.

### BRIEF DESCRIPTION OF THE DRAWINGS

The above-mentioned and other advantages and objects of this invention, and the manner of attaining them, will become more apparent, and the invention itself will be better understood, by reference to the following description of embodiments of the invention taken in conjunction with the accompanying drawings, wherein:

FIG. 1 is a perspective view of one embodiment of a pharmaceutical delivery device of the present invention in an initial or ready arrangement;

FIG. 2 is a front view of the pharmaceutical delivery device of FIG. 1;

FIG. 3 is a side view of the pharmaceutical delivery device of FIG. 1;

FIG. 4 is a longitudinal cross-sectional view of the device taken along line 4-4 of FIG. 2;

FIG. 5 is a longitudinal cross-sectional view of the device taken along line 5-5 of FIG. 3;

FIG. 6 is an exploded perspective view of the pharmaceutical delivery device of FIG. 1, but with the syringe and needle sterility-maintaining portions of the cap not shown;

FIGS. 7a and 7b are side and front views in longitudinal cross-section of the plunger sleeve of FIG. 6;

FIGS. 8a and 8b are side and front views in longitudinal cross-section of the syringe carriage of FIG. 6;

FIGS. 9a and 9b are side and front views in longitudinal cross-section of the outer housing body of FIG. 6;

FIG. 10 is a front view of the pharmaceutical delivery device of FIG. 1 after cap removal and being manipulated into a needle inserting arrangement;

FIG. 11 is a front view in longitudinal cross-section of the pharmaceutical delivery device of FIG. 10;

FIG. 12 is a front view of the pharmaceutical delivery device of FIG. 10 after being manipulated to deliver its contained pharmaceutical;

FIG. 13 is a front view in longitudinal cross-section of the pharmaceutical delivery device of FIG. 12;

FIG. 14 is a front view of the pharmaceutical delivery device of FIG. 12 after being manipulated into a needle retracted arrangement;

FIG. 15 is a front view in longitudinal cross-section of the pharmaceutical delivery device of FIG. 14;

FIG. 16 is a partial view in longitudinal cross-section of the pharmaceutical delivery device of FIG. 1 further illustrating a use staging feature;

FIG. 17 is a view similar to the view of FIG. 16 but after the cap has been rotated to be cammed out, and then pulled slightly axially;

FIG. 18 is a front view of another embodiment of a pharmaceutical delivery device of the present invention in an initial arrangement;

FIGS. 19a, 19b, 19c and 19d are front views of the delivery device of FIG. 18 after various stages of its manual operation;

FIG. 20 is a front view of the outer housing core body and gripping layer of the delivery device of FIG. 18;

FIG. 21 is a perspective view of the outer housing end plate of the delivery device of FIG. 18;

FIG. 22 is a perspective view of a needle cap portion of the delivery device of FIG. 18;

FIG. 23 is a front perspective view of the syringe carriage of the delivery device of FIG. 18;

FIG. 24 is a rear perspective view of the syringe carriage of FIG. 23, which carriage has assembled thereto a needle capped syringe with a capture collar and a compression collar;

FIG. 25 is a side view in longitudinal cross-section of the outer housing core body and gripping layer of FIG. 20;

FIGS. 26a and 26b are a perspective view and a front view in longitudinal cross-section of the plunger of the delivery device of FIG. 18;

FIG. 27 is a partial offset front view in longitudinal cross-section of the delivery device of FIG. 18, which cross-sectional offset front view is taken through a plane angularly offset eleven degrees from a front view;

FIG. 28 is a partial front view in longitudinal cross-section of the delivery device of FIG. 18;

FIG. 29 is a partial side view in longitudinal cross-section of the delivery device of FIG. 18;

FIG. 30 is a partial offset front view in longitudinal cross-section of the delivery device of FIG. 19a;

FIG. 31 is a partial front view in longitudinal cross-section of the delivery device of FIG. 19b;

FIG. 32 is a partial offset front view in longitudinal cross-section of the delivery device of FIG. 19c; and

FIG. 33 is a partial offset front view in longitudinal cross-section of the delivery device of FIG. 19d.

Corresponding reference characters indicate corresponding parts throughout the several views. Although the drawings represent embodiments of the present invention, the drawings are not necessarily to scale, and certain features may be exaggerated or omitted in some of the drawings in order to better illustrate and explain the present invention.

### DETAILED DESCRIPTION OF THE INVENTION

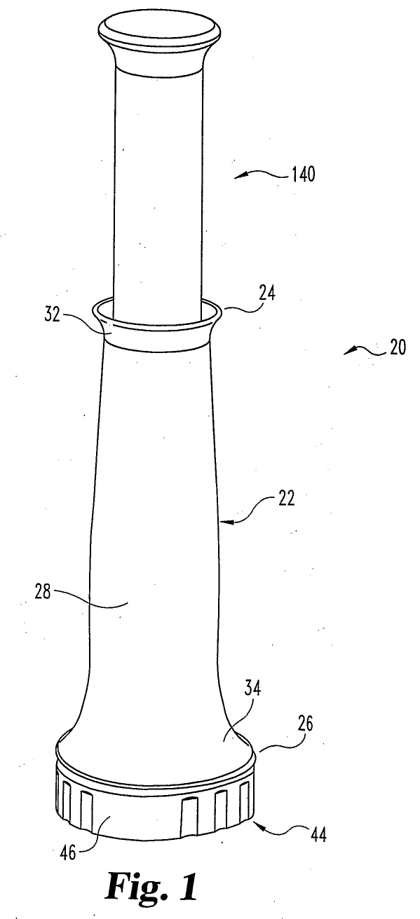

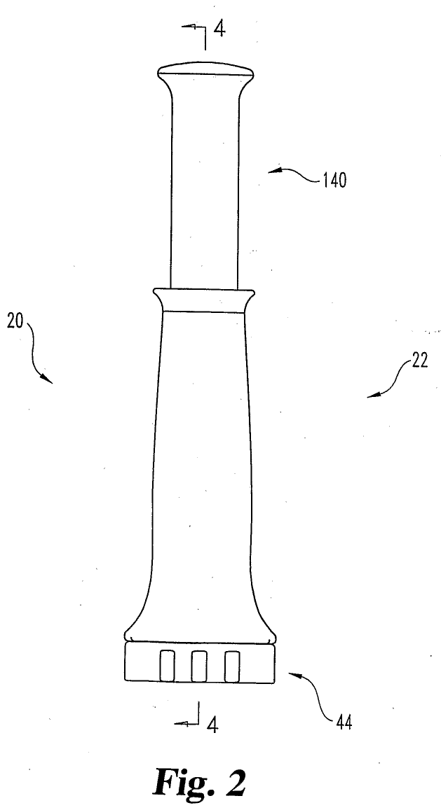

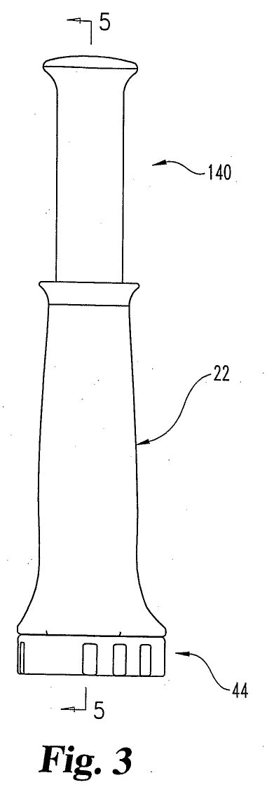

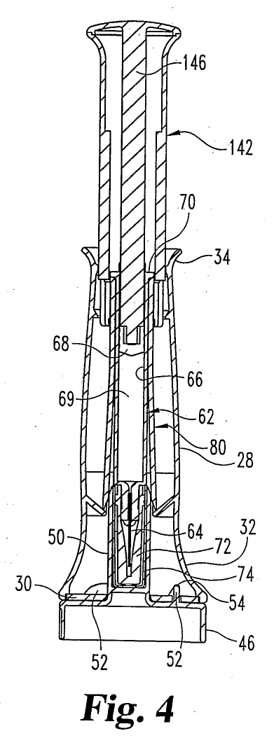

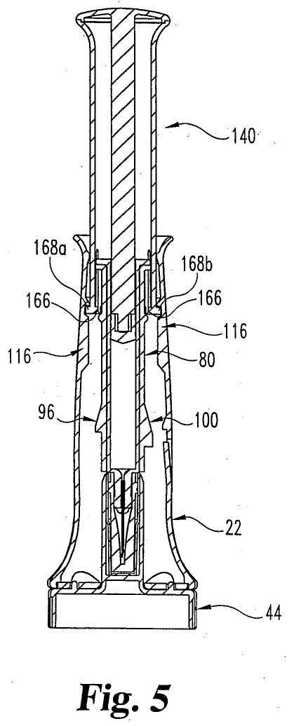

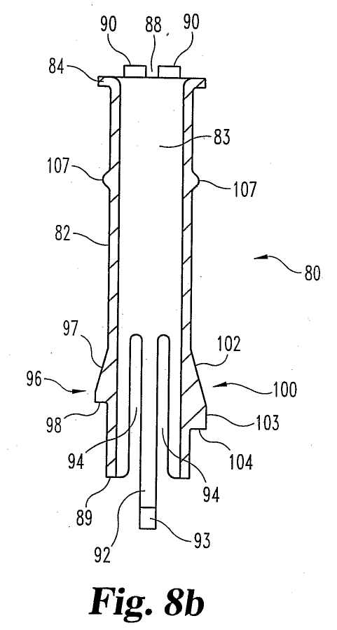

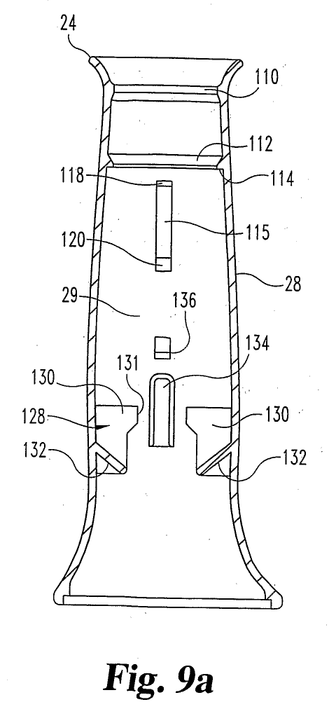

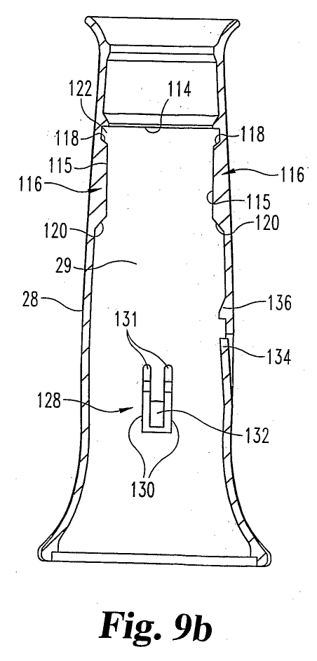

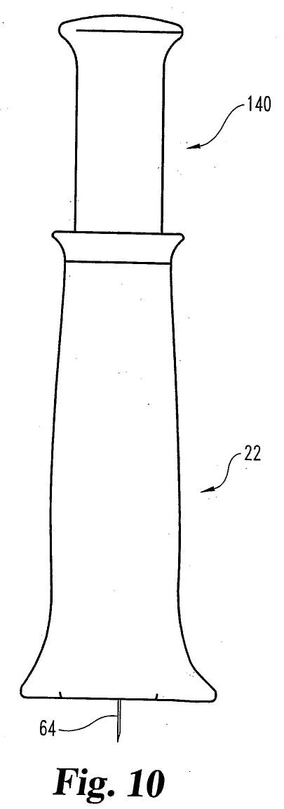

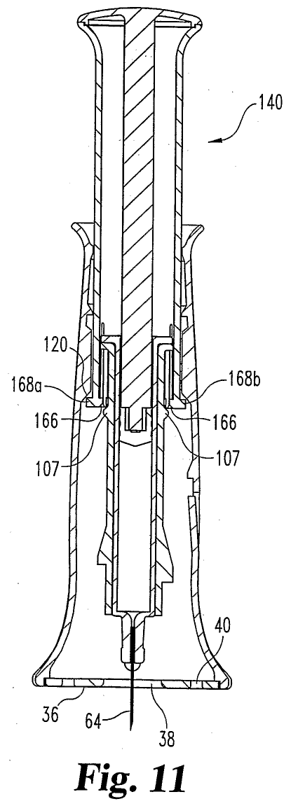

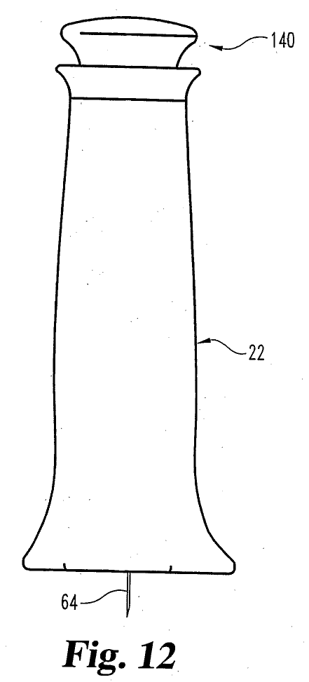

Referring now to FIGS. 1-17, there is shown one embodiment of a pharmaceutical delivery device of the present invention. The delivery device, generally designated 20, is a single use delivery device based on a standard, prefilled syringe as primary containment. The device is delivered ready to use, and is operable to provide a single, fixed dose delivery of its prefilled medication.

Any directional references in this detailed description with respect to the Figures, such as up or down, or top or bottom, are intended for convenience of description, and by itself does not limit the present invention or any of its components to any particular positional or spatial orientation.

Delivery device 20 is designed to allow a user, with one hand on the device, to comfortably position the device on the skin at a pre-selected site. After such siting, typically the user's other hand can be used to manually drive down the device plunger to cause needle insertion and drug delivery. Then, the user can withdraw the device plunger to retract the injection needle inside the device housing for its automatic locking.

Delivery device 20 includes an outer housing 22 having a distal end 24 and a proximal end 26. As used herein, distal and proximal refer to axial locations on the delivery device relative to an injection site when the device is oriented for use at such site, whereby, for example, proximal end of the housing refers to the housing end that is closest to such injection site.

The exterior periphery of outer housing 22 is sized, shaped and constructed of materials to facilitate being gripped within one hand by a user or a caregiver during site selection and injection, and may accommodate reduced dexterity in users such as rheumatoid arthritis patients. Outer housing 22 is shown formed by a tri-lobular body 28 and end plate 30 that are fixedly attached together. The distal region 32 of body 28 is flared slightly outward distally, and the body proximal region 34 is flared even farther outward to provide a wider base for stabilization. The flared distal region 32 may be used by some users to facilitate one-handed operation of the device, as such region may be gripped under by the fingers of a user who uses her thumb to plunge the device. The exterior of body 28 is overall shaped similar to that disclosed in U.S. Provisional Patent Application No. 60/695,048, which application is incorporated herein by reference in its entirety.

Body 28 and plate 30 may be formed of one or more materials. Clear or translucent plastics may be used to allow visibility of the device components housed therein. Although shown as being provided by a one-piece, plastic injection molded part, body 28 may be differently formed out of different materials, such as mating longitudinal sections that are assembled during manufacture around other components of device 20, or by axially mating tube sections, or by a combination thereof.

The proximal face 36 of plate 30 serves as the injection site skin-contacting surface of outer housing 22. Plate 30 defines a central, circular aperture 38, aligned with the longitudinal axis of device 20, through which an injection needle is moved from within housing 22 during needle insertion. At least one arcuate slot, and preferably a plurality of, such as three, arcuate slots 40, are disposed radially outward of aperture 38 and have a center of curvature aligned with the longitudinal axis of the device. The three slots 40 are equally angularly spaced, and each span about sixty degrees. Plate 30 may be provided with additional targeting guides, such as made of a high friction material for gripping an injection site.

A plastic needle cap 44 that covers aperture 38 is designed to be removed manually by a person having even limited dexterity or grip strength. Cap 44 includes a base 46 and an upstanding, blind bored cylindrical member 50 that projects distally from a planar face 48 of base 46 and fits through aperture 38. The exterior of base 46 is tri-lobular in shape and sized to have the same footprint as body proximal region 34. A series of grooves 47 in the periphery of the base 46 provide a knurled feature to facilitate gripping. Rather than or in addition to such knurled feature, base 46 may be provided with a high friction, readily grippable overmolding to facilitate gripping. A bevel 51 is formed at the outer rim of member 50.

Cap 44 includes at least one cam, and preferably a plurality of curved cams 52 such as the three shown, to provide a mechanical advantage that facilitates cap removal. Each cam 52 is in registry with a slot 40 when cap 44 is angularly oriented with the tri-lobular shapes of base 46 and body region 34 in alignment. The three cams 52 are angularly spaced in a circular configuration and project distally from base face 48. Each cam 52 includes an arch-shaped, distal camming face 54 functional with opposite slot ends 40a and 40b to provide a proximal shifting of the cap relative to the housing for either direction of cap twisting. Each cam 52 spans about sixty degrees, such that when cap 44 is mounted to the rest of the device 20 as shown in FIG. 1, a thirty degree rotation of cap 44 relative to outer housing 22 by the user causes camming face 54 to slide against the plate at a slot end (40a or 40b, depending on the direction of rotation) to thereby fully cam and shift cap base 46 away from plate 30. The rotational non-alignment of the tri-lobular base 46 and body region 34 resulting from employing the camming feature provides a visual indicator to the user that the cap 44 can be more easily pulled off completely.

The internal hollow 57 of cap cylindrical member 50 accommodates the injection needle 64 of the syringe 62, which syringe may be of known design. In the shown embodiment, syringe 62 includes a glass, tubular barrel 66 to which needle 64 is mounted, and an elastomeric stopper or piston 68. Piston 68 sealably slides within the medicine filled interior hollow 69 of barrel 66 into which needle 64 opens. The distal end of barrel 66 includes an asymmetrical, radially extending flange 70. The sterile needle 64 is covered by an elastomeric needle shield 72 and an overfitting plastic needle cover 74 that engages shield 72, which shield and cover function as part of cap 44. This shown syringe may be configured from the BD Hypak™ of Becton, Dickinson and Company. The needle cover 74 is engaged by cap cylindrical member 50, for example via a frictional fit, an adhesive connection, or a snap-fit connection, such that member 50 and the cover 74 and shield 72 are axially and rotationally fixed together. A user's manual removal of cap base 46 from the housing to prepare the device for use removes member 50, shield 72 and cover 74 from needle 64.

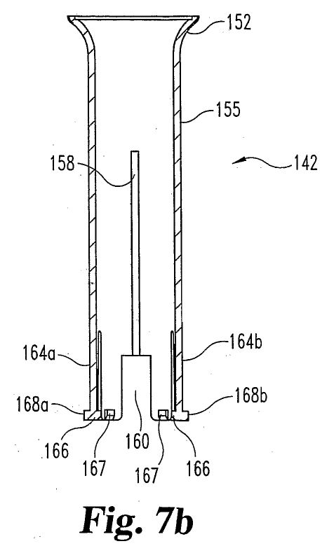

A syringe carriage 80 of device 20 is disposed within outer housing 22. Carriage 80 is rotatably fixed relative to outer housing 22, but is selectively axially movable therein to allow for device functionality as described further below. With additional reference to FIGS. 8a and 8b, carriage 80 includes a tubular body 82 with an outward projecting flange 84 at its distal end. The interior hollow 83 of body 82 accommodates syringe barrel 66 axially therethrough. Longitudinally extending ribs 86 extend from the underside of flange 84 to provide stability to help maintain alignment of the carriage in a plunger sleeve. A pair of diametrically opposed grooves 88 formed in the outer periphery of flange 84 serve as keyways. Pairs of lugs 90 are integrally formed with and project distally from flange 84. Lugs 90 are arranged to further define grooves 88, and also flank a distal facing ledge region 85 of flange 84 shaped to accommodate flange 70 of syringe 62. Carriage 80 and syringe 62 are axially and rotatably fixed together, such as via an adhesive connection between ledge region 85 and syringe flange 70. The keying provided by lugs 90 assists such adhesive connection by preventing rotation of flange 70 relative to carriage 80.

Projecting from its proximal end 89, carriage body 82 includes at least one staging finger, such as a pair of axially extending, flexible fingers 92 that are diametrically disposed. The inner face of each finger 92 includes a beveled end 93. Fingers 92 serve to stage the use of device 20 by frustrating attempts to inject prior to removal of cap 44. Each finger 92 is defined by a pair of parallel slots 94 that longitudinally extend distally from proximal end 89.

A first protuberance 96 and a second protuberance 100 are formed on the exterior of carriage body 82 nearer to the proximal end 89 than to the body distal end. Protuberances 96 and 100 are diametrically arranged on body 82.

First protuberance 96 serves as a latching rib and includes a distal surface 97 and a latching surface 98. Distal surface 97 ramps outward as it extends proximally. At the proximal end of surface 97, latching surface 98 extends radially inwardly therefrom.

Second protuberance 100 serves as a lock-supporting rib and includes a distal surface 102, a support surface 103 and an end surface 104. Distal surface 102 begins at the same axial position as distal surface 97 and also ramps outward as it extends proximally. At the proximal end of surface 102, support surface 103 extends axially therefrom, and end surface 104 extends radially inward from the proximal end of support surface 103.

Positioned distally of and in rotational alignment with protuberances 96, first and second detents 107 are formed on the exterior of carriage body 82. Detents 107 serve as push surfaces against which an axial force can be applied to carriage 80 to drive it proximally to cause needle insertion as described below. Fewer or additional push surfaces, having different shapes than the rounded detent shown, may be employed within the scope of the invention.

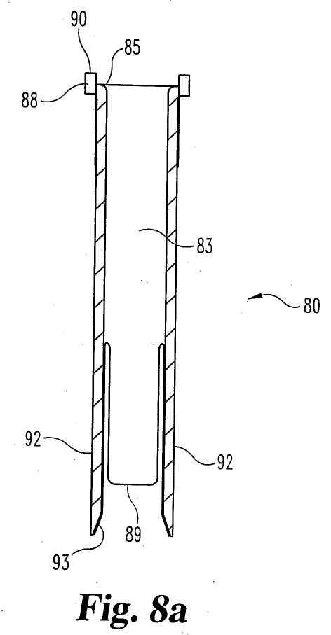

With additional reference to FIGS. 9a and 9b, the interior surface of housing body 28 defines an interior hollow 29 and is shown integrally provided with features that cooperate with carriage 80 and the plunger 140 of device 20. Axially spaced, circumferential ribs 110 and 112 are located near the distal end 24 of body 28 and center the plunger 140. The base of rib 112 is formed with a transversely oriented lip 114 that hinders plunger removal after assembly. A pair of diametrically opposed, longitudinally extending ribs, generally designated 116, are disposed proximally of rib 114. Each rib 116 includes a bar-shaped main section 115, a steep slope or ramp 118 leading to the distal end of section 115, and a shallow taper 120 at the proximal end of section 115. A space 122 defined between rib lip 114 and rib ramp 118 accommodate tabs 168a and 168b of the plunger 140. Ribs 116 cooperate with plunger 140 to achieve the proper syringe carriage movement during the needle insertion stage of plunging.

The body interior surface further includes at least one key module for engaging the carriage 80 to allow axial movement while preventing rotational movement relative thereto. In the shown embodiment, a pair of diametrically opposed key modules 128 are employed, each including a pair of parallel, longitudinal flanges 130 that terminate with radially inwardly projecting ears 131 at their distal ends. Ears 131 are sized and configured to slide within slots 94 of carriage 80. Ears 131 abut the distal ends of slots 94 when needle 64 is fully extended from housing 22 for injection during use.

A ledge 132 that provides an abutment or stop for finger 92 is formed into the body interior surface between the pair of flanges 130 of each key module 128. In a radially inward direction, each ledge 132 slopes slightly proximally, such as at about forty degrees from horizontal, to limit the possibility of a finger 92 being jammed against the ledge accidentally.

At an equal angular spacing between the key modules 128, a latching finger 134 formed integrally with housing body 28 projects radially inwardly and distally to be disposed at an angle within hollow 29. Finger 134 is used to lock the carriage and its held syringe in a retracted position, at which position needle 64 is fully disposed within housing 22, after use. Finger 134, due to housing body 28 being constructed from a resilient material, possesses flexibility allowing it to be cammed outward by, and then to resume an inward, latching position after passage of, a latchable protrusion of plunger 140. An inward stop lug 136 is formed in a close, axially spaced relationship with the distal end of finger 134, which spacing is sized to insertably accommodate the latchable protrusion.

The plunger of device 20 is generally designated 140 and is manually operable by a user to affect operation of the device. In the shown embodiment, plunger 140 is formed from a plunger sleeve 142 and a plunger stem 144 that are assembled together during manufacture. Such two-part construction, in addition to facilitating molding, allows materials with different properties to be used for the parts, such as a clear or translucent material for sleeve 142 allowing visibility of device regions enclosed therein, and an opaque material for stem 144.

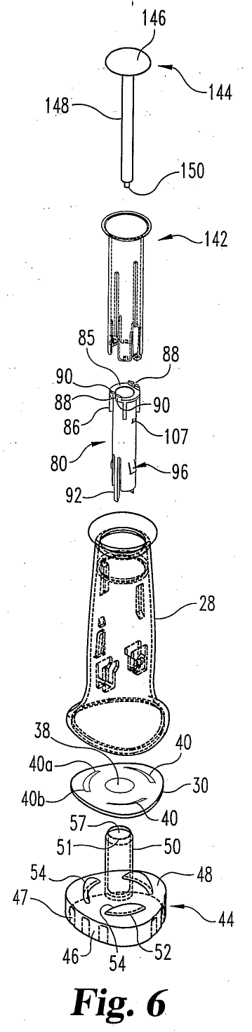

As shown in FIG. 6, stem 144 includes a convex distal head 146 from which depends an integrally formed shank 148. Head 146 is intended as the surface to be directly pressed on by a user to plunge the plunger. The proximal end 150 of shank 148 is shown with a stepped-down construction allowing insertion within the cavity of syringe piston 68, but such construction is not necessary to the invention.

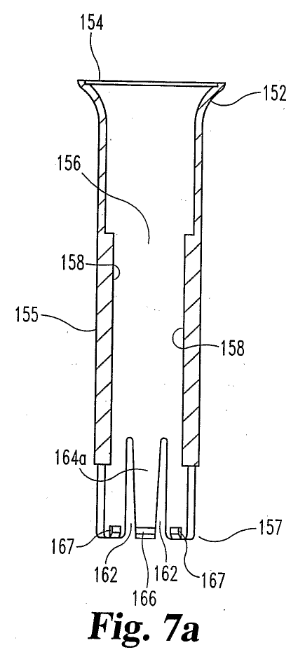

With additional reference to FIGS. 7a and 7b, plunger sleeve 142 includes a flared, open distal end 152 provided with an annular lip 154. Plunger head 146 seats on lip 154 and is secured thereto, such as via an ultrasonic weld or a not shown snap-fit attachment. The flaring of end 152 allows for a ready gripping of the plunger by a user for withdrawing the plunger so as to retract the needle after injection. The cylindrical body 155 of sleeve 142 defines an interior hollow 156 having inwardly projecting, longitudinally extending keys 158 that fit within carriage grooves 88 to rotatably fix together carriage 80 and plunger 140. A pair of upwardly extending diametrically opposed notches 160 is formed in the proximal end 157 of body 155. Notches 160 fit around key modules 128 when the plunger 140 is fully plunged during injection. Two pair of slots 162 extending from proximal end 157 define diametrically opposed resilient prongs 164a and 164b. Each of prongs 164a and 164b includes at its proximal end an inwardly projecting tab 166. The sleeve body sections formed between notches 160 and slots 162 each include an inward tab 167 at its proximal end. Tabs 167 abut the underside of ribs 86 to locate carriage 80 relative to plunger 140. Prongs 164a and 164b further include, respectively, radially outwardly projecting tabs 168a and 168b at their proximal ends. Tabs 168a and 168b slide along body ribs 116 during the plunging of plunger 140 relative to the housing 22 from the ready position shown in FIG. 1, which sliding motion, due to their complementary shapes, brings plunger 140 into and then out of driving engagement with carriage 80.

In particular, when device 20 is arranged in its ready condition shown in FIG. 1, tabs 168a and 168b are disposed within space 122. As plunger 140 is initially pushed down by a user relative to housing 22, tabs 168a and 168b engage ramps 118, and further proximal motion of plunger 140 results in these tabs sliding along and being cammed radially inward by ramp 118, thereby bending prongs 164a and 164b inward such that their tabs 166 close the radial gap with the carriage body 82. The steep slope of ramp 118 is selected to provide a suitably large resistance that serves as a clear decision point for a user as to when to start an insertion, and to further promote a plunger acceleration that causes a smooth needle injection. Until this point, carriage 80 has not been shifted by movement of plunger 140. After sliding along ramps 118, tabs 168a and 168b slide along rib sections 115 as plunging continues. During this motion, the tabs 166 of the prongs 164a and 164b drivingly abut detents 107 of the carriage 80. Because rib sections 115 prevent these prongs from splaying outward, plunger sleeve 142, as it continues to be shifted manually proximally, also shifts proximally the syringe holding carriage 80, causing needle 64 to extend through aperture 38 of plate 30 and penetrate the injection site. Carriage advancement continues with plunger advancement until the ends of slots 94 reach housing ears 131, at which point tabs 168a and 168b have reached rib tapers 120 and are free to shift outward such that their respective tabs shift radially outward to disengage from detents 107. The provision of tapers 120 may limit noise when the tabs shift outward which might confuse a user. Thus, as a result, further axial plunging of plunger 140 does not drive carriage 80, but instead, due to the positioning of plunger shank 148, drives syringe piston 68 proximally within barrel 66 to expel medication from reservoir 69 through needle 64 and into the injection site.

At the point the injection is completed, plunger 140 has reached a plunged position where tab 166 of finger 164a has ramped over rib surface 97 and has snapped inward such that it is disposed proximally of latching surface 98. Meanwhile, tab 166 of finger 164b has slid along rib surface 102 and is disposed along support surface 103, which surface is sized such that prong 164b is bent outward to a position such that its tab 168b is disposed radially outward of the inner reach of the distal tip of finger 134.

When the plunger 140 with the now latched carriage is manually retracted distally by the user, tab 168b will cam outward finger 134 until it reaches a position thereabove, at which point finger 134 snaps back inward, thereby capturing tab 168b axially between lug 136 and finger 134 to limit any further axial movement of the carriage and therefore needle 64.

The construction of device 20 will be further understood in view of the following explanation of an exemplary operation. The user will typically be provided with a device in its capped, ready state as shown in FIGS. 1-5. The cap 44 including its held shield 72 and cover 74 are first removed by being shifted proximally relative to housing 22, which removal leaves injection needle 64 entirely within the confines of body 28. Such shifting may be aided by the user twisting cap base 46 relative to housing 22, which twisting causes camming faces 54 to slide along slot ends 40a or 40b to force the cap proximally.

Next, the outer housing 22 is gripped in the hand of a user and placed with end plate 30 against the injection site. The translucent construction of body 28 allows aperture 38 to be visible around the site. Then, while continuing to hold the outer housing, the user, typically with the palm of the other hand, applies a plunging force directly on plunger head 146. As this force is applied, plunger 140 starts to move proximally, until as described above, tabs 166 of prongs 164a and 164b abut push surfaces 107, such that as the plunger continues to be manually driven downward, carriage 80 and its held syringe 62 are simultaneously, and in an equal amount, driven proximally, which motion causes injection needle 64 to pass through aperture 38 and into the user at the injection site. Further plunging of plunger 140 advances piston 68 within syringe barrel 66 to force the contained medicine out through the injection needle 64.

After the medication injection, device 20 allows a user to manually retract the needle within the housing for disposal of the device. By gripping the outer housing body 28 in one hand and the plunger 140 with the other, and then pulling these pieces apart, plunger 140 is withdrawn from within body 28. Due to the plunger's latching of the carriage via the engagement of carriage surface 98 by tab 166 of prong 164a, carriage 80 and its held syringe 62 are shifted distally into housing 28. Such retraction can continue until tab 166 of prong 164b is captured between locking finger 134 and lug 136, at which point the now fully retracted needle is completely enclosed within the housing 28 and prevented from further motion.

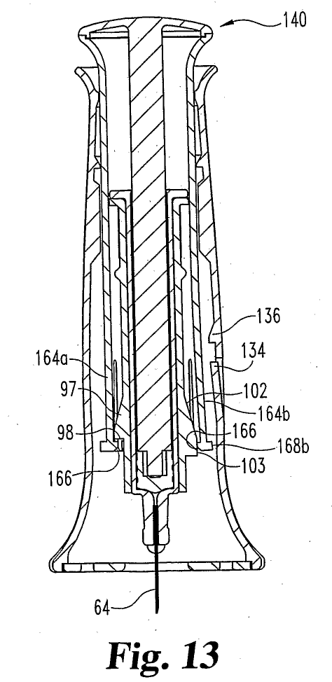

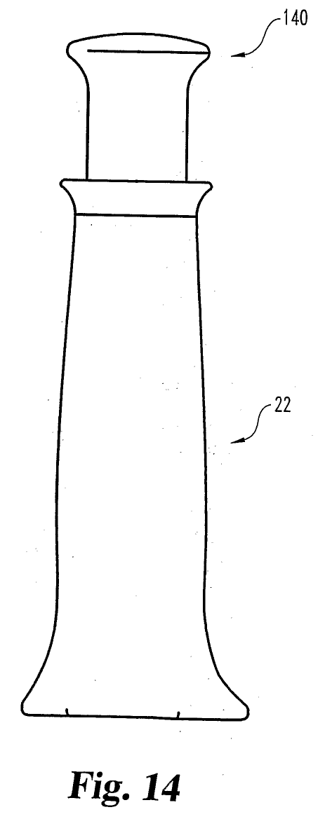

If a user attempts to plunge plunger 140 relative to housing 22 so as to cause an injection prior to removal of cap 44 from outer housing 22, such attempt will be frustrated by the interaction of cap 44 with carriage 80. In particular, as shown in FIG. 16, when cap 44 is fully installed on device housing 22, cap member 50 engages and splays outward fingers 92 of carriage 80. The bevels 51 and 93 facilitate this engagement during assembly. When so splayed outward, fingers 92 prevent proximal motion of carriage 80, and therefore the syringe needle 64, due to the abutment of the tips of fingers 92 against ledges 132. Only after the cap 44 has been removed such that cap member 50 is withdrawn from between the fingers 92 are such fingers free to return to their neutral state radially inward of ledges 132 as shown in FIG. 17, at which state carriage advancement is no longer prevented by the fingers 92.

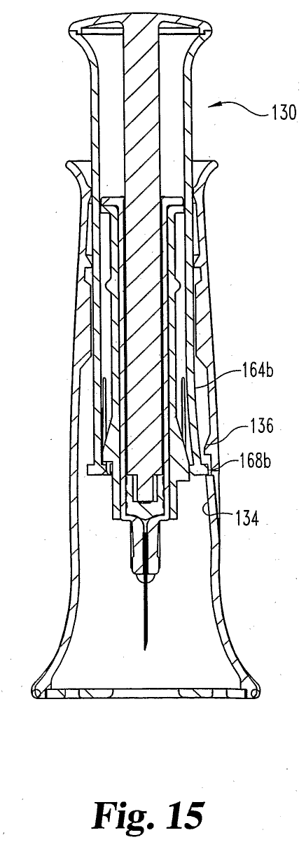

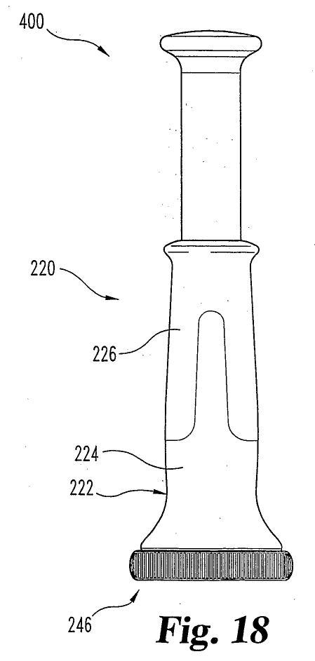

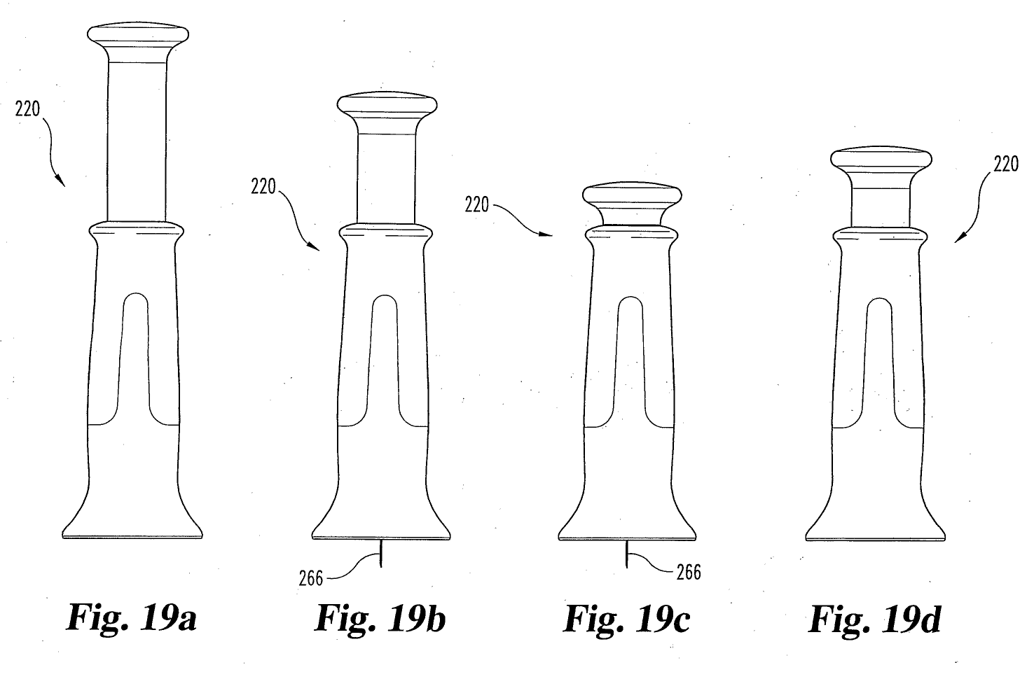

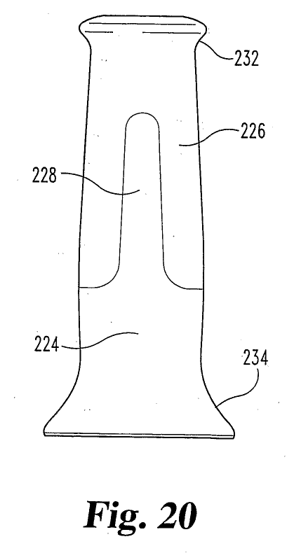

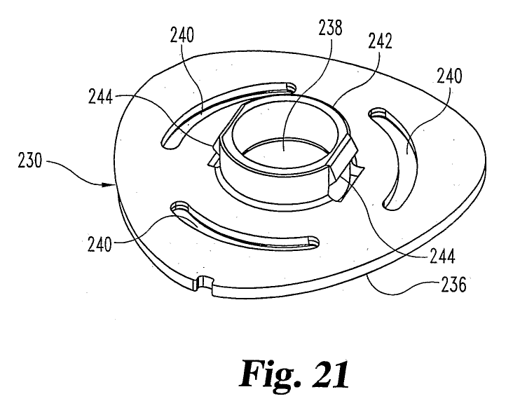

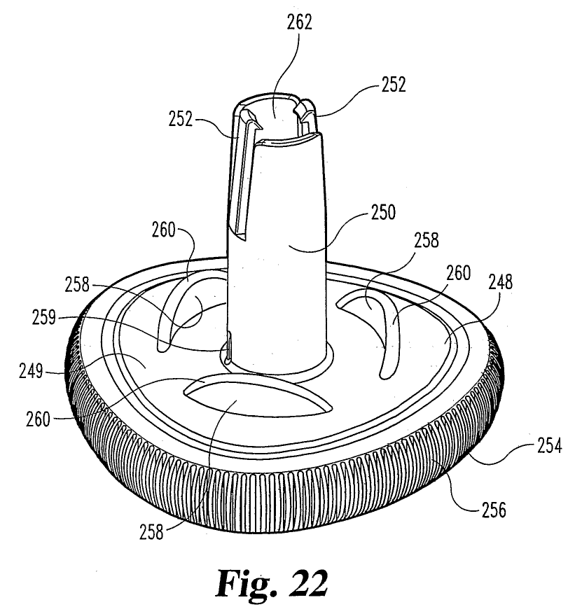

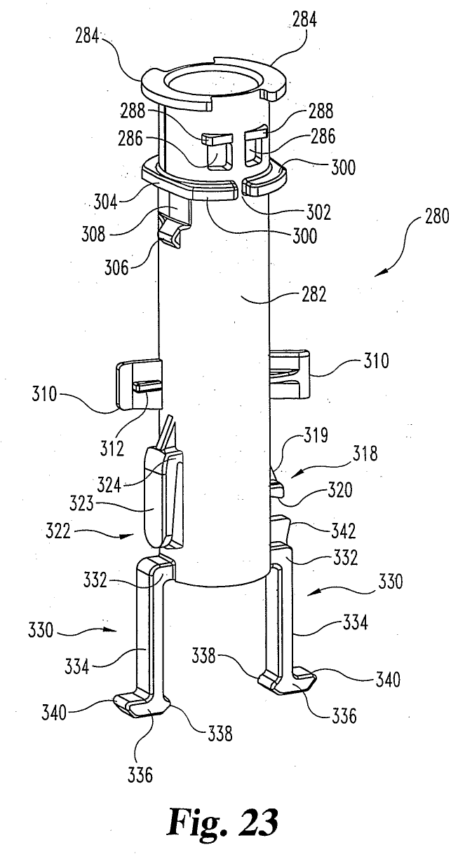

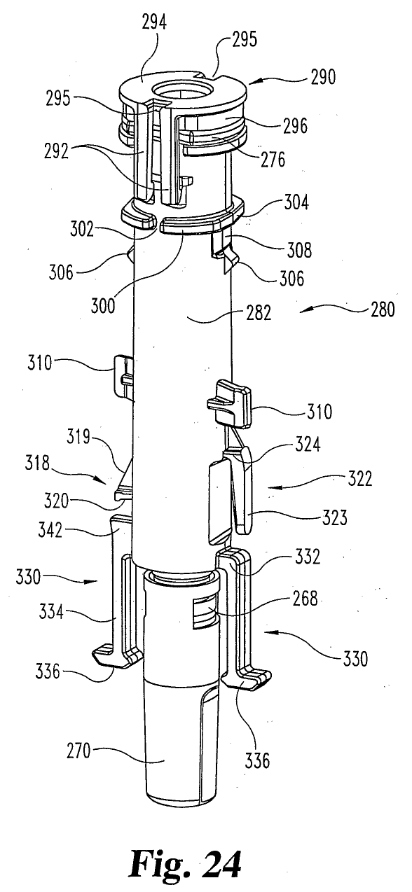

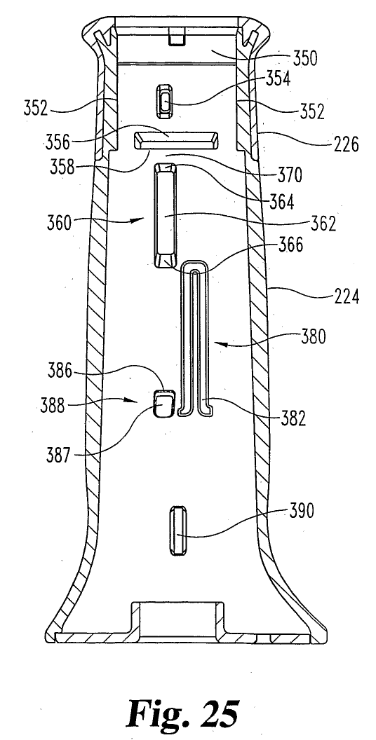

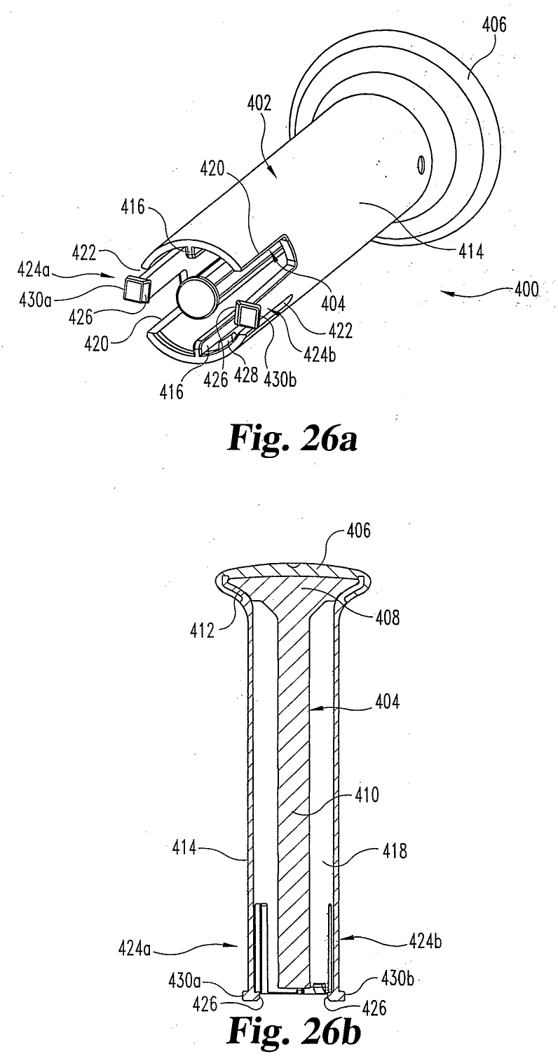

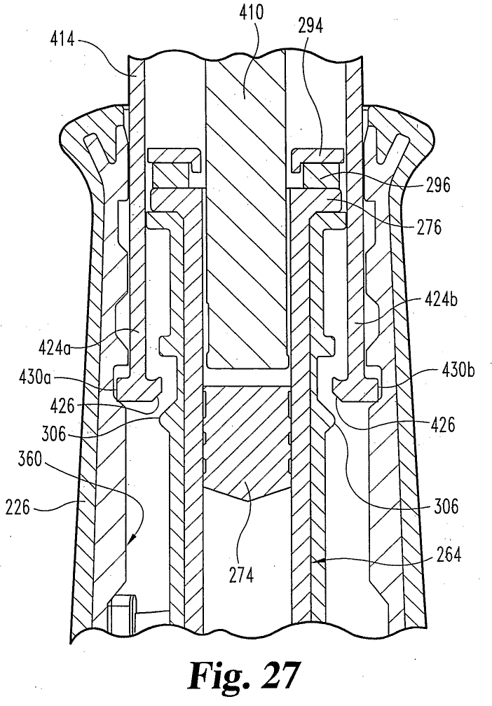

Referring now to FIGS. 18-33, there is shown another embodiment of a single use pharmaceutical delivery device of the present invention, which device is generally designated 220. Device 220, in many respects, is similar in overall design and operation to device 20, but certain differences will be apparent to one skilled in the art from the following description.

The outer housing 222 of delivery device 220 is sized, shaped and constructed of materials to facilitate being gripped within one hand by a user or a caregiver during site selection and injection. Outer housing 222 includes a core body 224 molded in one piece from a transparent plastic, as well as a gripping layer 226 that is overmolded to core body 224. Gripping layer 226 is made of an opaque material, which material is a resilient or soft touch material such as a thermoplastic rubber to promote a ready grip. Gripping layer 226 is shaped to expose a pair of diametrically opposed channel sections 228 of core body 224 which allow visibility of the medicine within the device. Outer housing 222 also includes an end plate 230 that is fixedly attached over the open proximal end of core body 224. End plate 230 is formed of a transparent plastic to promote visibility, such as of the injection site.

The majority of the longitudinally extending length of outer housing 222 is shaped to be generally circular in transverse cross-section. The distal region 232 of outer housing 222 is flared slightly outward distally in that circular shape. The proximal region 234 of outer housing 222 is flared even farther outward and with a tri-lobular shape to provide a wider base for stabilization, easier gripping and an anti-roll feature.

End plate 230 has a flat, injection site skin-contacting surface 236, and defines a central, circular aperture 238, aligned with the longitudinal axis of device 220. Three arcuate slots 240 are disposed equally radially outward of aperture 238. Slots 240 are equally angularly spaced, and each span about eighty degrees and have a center of curvature aligned with the longitudinal axis of the device. A collar 242 formed integrally with and projecting from the distal face of plate 230 rings aperture 238. Two detenting features 244 are formed on the outer radial periphery of collar 242 at diametrically opposed locations. Additional targeting guides are not shown but may be provided on end plate 230.

A manually removable needle cap 246 that when mounted covers all of end plate 230 and thereby aperture 238 includes a tri-lobular base plate 248 and an upstanding, cylindrical member 250. Plate 248 and member 250 are molded from plastic as a single piece, which piece may be formed of an opaque material to visually distinguish the piece from the device body, or which piece may be formed of a different, such as transparent, material. Member 250 projects distally from a planar face 249 of base plate 248 and fits through aperture 238. The distal end of member 250 is longitudinally slotted to define a pair of resilient latching fingers 252 for needle shield removal. The radial periphery of base plate 248 is overmolded with a gripping feature 254 made of a soft touch material and which has a series of longitudinally extending ribs 256 to be easily grippable. The base plate with the overmolded gripping feature is sized and shaped to have a footprint that is a flared extension of the footprint of body proximal region 234. Diametrically disposed ribs 259 at the base of member 250 provide an interference with the inner radial surface of collar 242 to stabilize the cap during shipping and handling, thereby limiting force applied to the needle shield prior to intentional cap removal.

Cap 246 includes three cams 258 that are each in registry with a different slot 240 when cap 246 is angularly oriented in alignment with the tri-lobular shapes of base plate 248 and proximal region 234. The three cams 258 are angularly spaced in a circular configuration and project distally from base face 249. Each cam 258 includes an arch-shaped, distal camming face 260 that spans about eighty degrees, which camming face is functional with opposite slot ends to provide a proximal shifting of the cap relative to the housing for either direction of cap twisting.

The internal hollow 262 of cap cylindrical member 250 accommodates the injection needle 266 of the syringe 264, as well as the elastomeric needle shield 268 and overfitting plastic needle cover 270 that engages shield 268, which shield and cover function as part of cap 246. During manufacturer mounting of the cap over the proximal end of the outer housing, latching fingers 252 snap fit over the distal end of the needle cover 270. Fingers 252 serve to pull the needle cover, and thereby the shield that is engaged by the cover, off the needle 266 when the cap 246 is turned and pulled from the rest of device 220. Syringe 264 is similar to syringe 62, and includes a glass barrel 272, a sealing piston 274, and a bilaterally symmetrical flange 276.

A syringe carriage 280 is made of a transparent plastic and includes a tubular body 282 with a pair of angularly spaced, outward projecting flanges 284 at its distal end that are diametrically disposed and which serve as a seat for syringe flange 276. The hollow interior of body 282 accommodates syringe barrel 272 therein. Four openings 286 each with a lip 288 at its distal end allows for a snap-fit engagement with two pairs of resilient latching fingers 292 depending from an annular plate 294 of a capture collar 290 made from plastic. Diametrically disposed notches 295 are provided on the outer radial periphery of plate 294. Fingers 292 fit within the angular space between the carriage flanges 284. An apertured compression collar 296 made of a cellular urethane foam fits in the axial space between syringe flange 276 and capture collar 290 and accounts for syringe tolerances. Syringe 264 is thereby axially and rotatably fixed with carriage 280.

Proximal of openings 286, a pair of angularly spaced ribs 300 ring the exterior of body 282 and define a pair of diametrically opposed gaps 302. Gaps 302 are in alignment with notches 295 as well as the angular space between the fingers of each pair of latching fingers 292. Diametrically opposed portions of ribs 300 are flattened at 304 to allow passage of plunger staging arms or prongs described further below.

Proximal of ribs 300, first and second detents or jams 306 are formed on the exterior of carriage body 282. Detents 306 are diametrically positioned and serve as push surfaces against which an axial force can be applied to carriage 280 to drive it proximally. A slight depression 308 is formed in body 282 between each detent 306 and rib flat 304 to further accommodate the plunger staging arms.

A pair of carriage guides 310 radially project from the exterior of body 282 proximally of, but angularly offset from, detents 306. Guides 310 are diametrically opposed. Each guide 310 includes a strengthening rib 312 that cooperates with the track in which its guide 310 slides to aid in centering carriage 280.

A first protuberance or latch element 318 and a second protuberance or springboard 322 are positioned proximally of guides 310. Latch element 318 and springboard 322 are diametrically arranged and formed on the exterior of carriage body 282 in angular alignment with jams 306. Latch element 318 includes a ramping distal surface 319 and a latching surface 320. Springboard 322 provides a resilient lock-supporting rib 323 that is cantilevered and depends proximally from a base portion 324 that projects radially from the carriage body 282.

Depending from its proximal end, carriage body 282 includes a pair of resilient staging or detent arms, generally designated 330. Detent arms 330 are diametrically disposed and each includes a base portion 332, an axially extending and flexible segment 334, and a foot 336 including an inwardly projecting clicker 338 and an outwardly projecting boss 340. The center of detent arms 330 is angularly offset by eleven degrees from the center of jams 306. The base portion 332 of one detent arm 330 includes an axially upward extending shoulder 342 that serves as a back-up hard stop for the device plunger. The shown detent arms 330 work with protrusions 390 to stage the use of device 220 by frustrating attempts to inject prior to removal of cap 246, to provide an initial resistance to carriage proximal movement, and to work with end plate detenting features 244 to provide a tactile and audible notice of the end of needle insertion during use, and further aid in the device being properly operated by a user.

The interior surface of core body 224 includes a stepped-in bearing region 350 for supporting and centering the device plunger. Bearing region 350 rings the body hollow interior and has depending therefrom a pair of diametrically opposed portions that serve as longitudinal ribs 352. A pair of diametrically opposed, longitudinally extending ribs 354 help to prevent buckling of the plunger. Another pair of diametrically opposed ribs 356 is located proximal of ribs 354. Each rib 356 extends only a portion of the core body inner circumference. The base of each rib 356 is formed with a transversely oriented lip 358 that hinders plunger removal after assembly. A pair of diametrically opposed, longitudinally extending ribs, generally designated 360, are disposed proximally of ribs 356. Each of ribs 360 includes a bar-shaped main section 362, a slope or ramp 364, such as about forty degrees from horizontal, leading to the distal end of section 362, and a taper 366, such as about fifty degrees from horizontal, at the proximal end of section 362. These angles of the slope and taper are merely illustrative, as different angles which provide for a transition without jamming of the plunger arms can be used. The axial space 370 defined between each rib lip 358 and each rib ramp 364 accommodate tabs 430a and 430b of the plunger 400.

Angularly offset from ribs 360 are a pair of diametrically opposed tracks 380 that cooperate with the carriage guides 310 to allow axial movement of the syringe carriage along the housing while preventing rotational movement relative thereto. Tracks 380 are each formed by a rib 382 that runs in an inverted U-shaped pattern, and each guide 310 slides within a channel defined by one of the ribs 382. The radially inward face of rib 382 is abutted by the outward face of guide ribs 312 for centering the carriage 280 within the housing.

The housing core body also includes a single stop ledge 388 projecting within the hollow. The stop ledge 388 has a transversely oriented distal face 386, a ramped proximal face 387, and is angularly aligned with one of the ribs 360. Stop ledge 388 is used to lock the carriage and its held syringe in a retracted position after use, at which position the needle is fully disposed within the housing.

Near the proximal end of core body 224, a pair of diametrically opposed protrusions 390 project from the core body within the hollow. Protrusions have angled distal and proximal surfaces to provide a detenting function with the detent arms 330 to stage device use.

The plunger of device 220 is generally designated 400 and includes a plunger sleeve 402 made from a transparent plastic, a plunger stem 404 made of an opaque plastic, and an overmolded, soft touch gripping layer 406 for user comfort at the distal end of the plunger.

Stem 404 includes a distal head 408 from which depends an integrally formed shank 410, the proximal end of which is intended to drivingly abut the syringe piston. Plunger head 408 is secured to a lip 412 of the flared distal end of sleeve 402 via a snap fit, and further secured via the overmold. The cylindrical body 414 of sleeve 402 defines an interior hollow 418 in which shank 410 is centered. Body 414 has inwardly projecting, longitudinally extending keys 416 that fit within plate notches 295 and gaps 302 to rotatably fix together carriage 280 and plunger 400.

A pair of upwardly extending, diametrically opposed notches 420 formed in the proximal end of sleeve body 414 fit around carriage guides 310 when the plunger 400 is fully plunged during injection. Notches 420 together with additional slots 422 define diametrically opposed, resilient staging arms or prongs 424a and 424b. Each of prongs 424a and 424b includes at its proximal end an inwardly projecting tab 426. The sleeve body also includes two diametrically arranged tabs 428 that abut the underside of ribs 300 to serve as a one-way snap during assembly of the carriage 280 and the plunger 400, and to limit any upward travel of the plunger relative to the carriage. Prongs 424a and 424b further include, respectively, radially outwardly projecting tabs 430a and 430b at their proximal ends. Tabs 430a and 430b slide along body ribs 360 during the plunging of plunger 400 to bring the plunger into and then out of driving engagement with carriage 280 similar to the manner described with respect to the embodiment of FIG. 1, except that the plunger prongs are not used as the primary means of providing an initial resistance to carriage movement, which resistance is provided by the interaction of detent arms 330 with protrusions 390

The construction of device 220 will be further understood in view of the following explanation of an exemplary operation. First, and with the device arranged as shown in FIGS. 18, 27 and 28, the needle cap 246 including its held shield 268 and cover 270 are removed from the syringe needle, such as by twisting and then pulling cap 246 proximally from the outer housing.

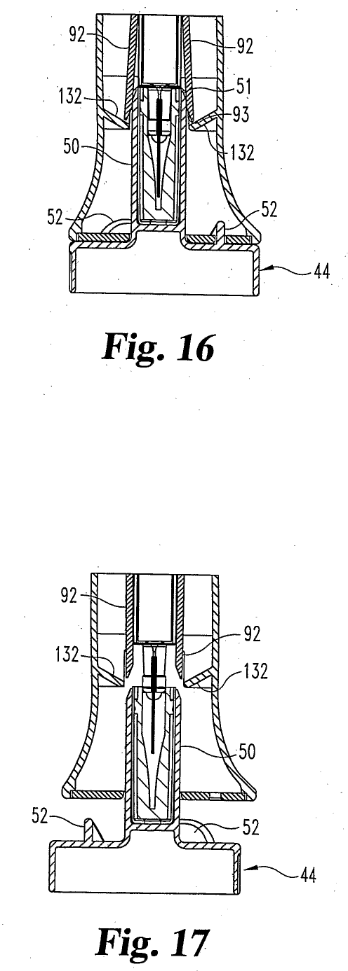

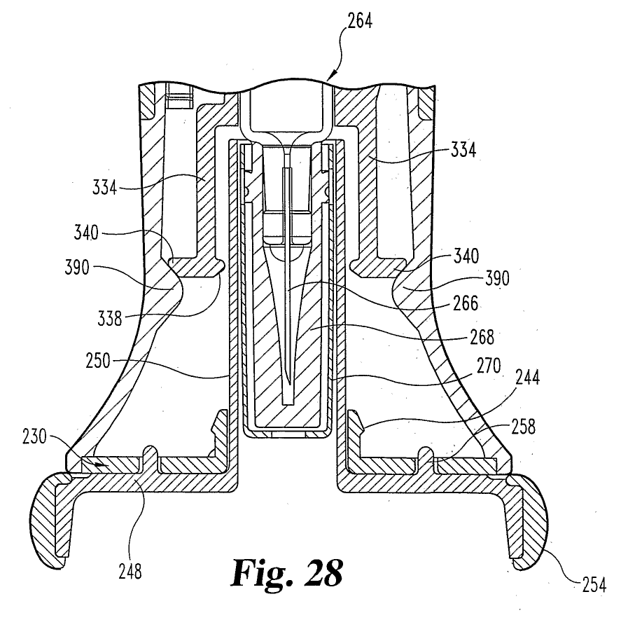

Next, after the user properly positions device 220 with end plate 230 against the injection site, the user applies a plunging force on plunger 400. As this force is applied, plunger 400 starts to move proximally, causing tabs 426 of prongs 424a and 424b to abut jams 306 due to the inward motion of the prongs resulting from the sliding of tabs 430 along body ramps 364. At this point, device 220 is arranged as shown in FIGS. 19a and 30.

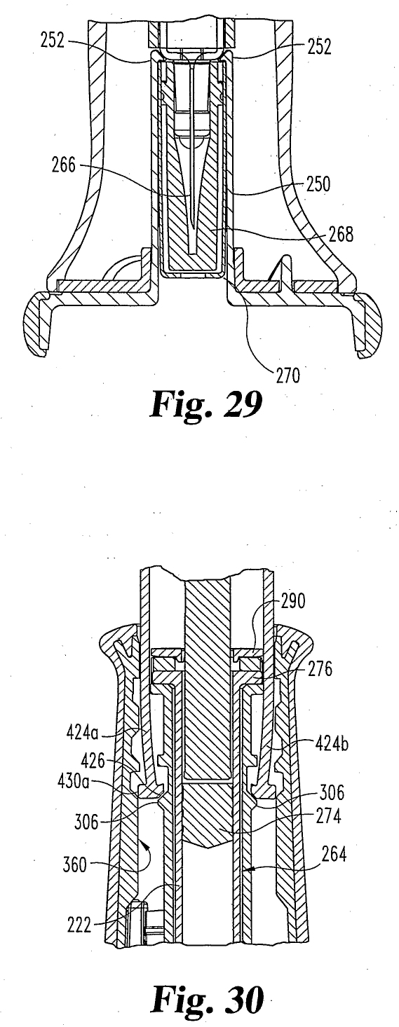

A further manual plunging of plunger 400 starts to drive carriage 280 and its held syringe 264 simultaneously, and in an equal amount, proximally. However, this further motion is initially resisted by the engagement of detent arms 330 with protrusions 390, as not until sufficient force is applied to the plunger to cause the arms to bend inward as bosses 340 slide along and over the protrusions 390 will the carriage be shifted farther proximally. It will be appreciated that if a user tries to plunge the device plunger prior to the removal of needle cap 246, such efforts will be unsuccessful as bosses 340 can not slide over protrusions 390 due to cap member 250 blocking clickers 338 of feet 336 from moving sufficiently radially inward. As the manual plunging continues, causing injection needle 266 to pass through aperture 238 and into the user at the injection site, the carriage 280 continues to move proximally until clickers 338 snap over detenting features 244, at which time the plunger staging arms 424a and 424b resiliently splay out of engagement with jams 306 due to tabs 430 reaching taper sections 366 of camming ribs 360. The snapping movement of the clickers 338 over the detenting features 244 provides a tactile and audible notice to the user of the end of needle insertion. At this point, as the feet 336 of the detent arms 330 bottom out on the end plate, further carriage proximal movement is halted, and device 220 is arranged as shown in FIGS. 19b and 31.

As a user continues to manually plunge the plunger 400 proximally, piston 274 within syringe barrel 272 is forced by plunger stem 404 to move proximally, forcing the medicine contained with the syringe out through injection needle 266. As plunger 400 is so moved proximally, prong 424a and its tab 430 freely pass radially inward of stop ledge 388.

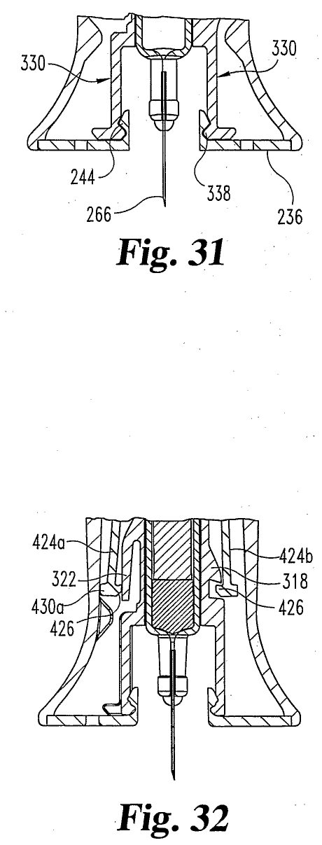

A user may continue plunging the plunger 400 until the full dose is administered from device 220, at which time the plunger 400 is mechanically halted, such as by piston 274 bottoming out within syringe barrel 272, or by the proximal end of prong 424b abutting carriage shoulder 342, or by the distal ends of notches 420 abutting the distal faces of carriage guides 310. As the plunger stroke was nearing its end, tab 426 of prong 424b has ramped over rib surface 319 and has snapped inward such that it is disposed proximally of latching surface 320. Meanwhile, tab 426 of prong 424a has slid along locking rib 323 of springboard 322 such that prong 424a is bent radially outward. At this point, device 220 is arranged as shown in FIGS. 19c and 32.

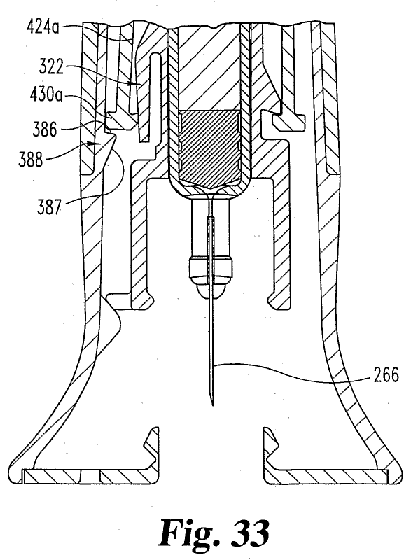

To manually retract the needle 266 within the housing, which retracting can occur either before or after the user pulls housing 222 such that the end plate 230 is lifted from the injection site, the plunger 400 is pulled up relative to the housing 222. This plunger motion, due to the carriage being latched thereto by the engagement of prong 424b with latching surface 320, shifts the carriage 280 with syringe 264 distally. Sufficient force must be applied to pull clickers 338 of the detent arms free from detenting features 244. As the carriage moves distally, and after the needle tip enters the housing interior, tab 430a of prong 424a reaches and slides along the ramped face 387 of stop ledge 388, which bends locking rib 323 of springboard 322 inward. When tab 430a has ramped over the stop ledge 388, locking rib 323 springs outward so as to snap tab 430a outward to be disposed proximally of the ledge. Further distal motion of the plunger 400 and the carriage 280 is prevented as guides 310 abut the top of tracks 380. The abutting engagement of tab 430a with ledge distal face 386 results in plunger 400 being locked from subsequent proximal plunging, thereby locking device 220 with the needle retracted within the housing. At this point, device 220 is arranged as shown in FIGS. 19d and 33.

While this invention has been shown and described as having preferred designs, the present invention may be modified within the spirit and scope of this disclosure. For example, while the embodiment of FIGS. 18-33 includes all of a feature preventing plunger plunging prior to cap removal, a feature by which the plunger resilient prong is brought into driving engagement with the syringe carriage, and a feature by which the carriage and its held syringe can be retracted and locked by pulling the plunger manually, each of, and any combinations of, these features may be employed in alternate embodiments. This application is therefore intended to cover any variations, uses or adaptations of the invention using its general principles. Further, this application is intended to cover such departures from the present disclosure as come within known or customary practice in the art to which this invention pertains.

We claim:

1. A pharmaceutical delivery apparatus comprising:
a housing extending between a distal end and a proximal end;
a syringe assembly including a needle having a proximal tip, said assembly plungeable relative to said housing from a first position, at which said needle tip is disposed within said housing, to a second position, at which said needle tip projects from said housing beyond said proximal end for insertion into an injection site;
a needle cap including a base and a stem, said base exposed at said housing proximal end to be manually grippable for cap removal, said stem upstanding from said base and sized and configured to insert through an opening in said housing proximal end to cover said needle tip when said syringe assembly is disposed in said first position; and
said needle cap base further including a plurality of distally projecting cams located radially outward of said stem, said cams fittable within slots in said housing proximal end when said cap is fully mounted to the apparatus, said cams and slots complementarily sized and configured whereby twisting of said fully mounted cap relative to said housing causes said cams to cammably slide along surfaces of said housing proximal end to shift said cap proximally relative to said housing.

2. The pharmaceutical delivery apparatus of claim 1 wherein a radial periphery of said housing proximal end and of said needle cap base are non-circular and correspondingly shaped, wherein said cams and slots are located such that the peripheries of said needle cap base and said housing proximal end are aligned when said cap is fully mounted to the apparatus, but wherein said peripheries are not aligned after the cam utilizing twisting of said cap.

3. The pharmaceutical delivery apparatus of claim 1 wherein said slots include arcuate slots formed through a plate-shaped housing proximal end, said slots angularly spaced in a circular arrangement that is centered on said opening.

4. The pharmaceutical delivery apparatus of claim 1 wherein said syringe assembly comprises a carriage for a needle-equipped syringe, said carriage having at least one proximally extending, resilient locking finger, wherein when said syringe assembly is disposed in said first position and said cap is fully mounted to the apparatus, said locking finger is bent outward by direct engagement with said cap stem, and wherein said housing comprises a stop surface for abutment by said bent outward locking finger to prevent shifting of said syringe assembly to said second position.

5. The pharmaceutical delivery apparatus of claim 1 wherein said syringe assembly comprises a manually shiftable plunger, and a carriage for a needle-equipped syringe, said plunger including at least one resilient prong disposed within said housing, and wherein said housing includes at least one camming rib for engagement with said prong, said carriage including at least one push surface on an exterior periphery, said camming rib structured and arranged to bias said resilient prong inward to engage said at least one push surface during proximal plunging of said plunger to affect proximal shifting of said carriage.

6. The pharmaceutical delivery apparatus of claim 5 wherein said at least one prong comprises a first prong and a second prong.

7. The pharmaceutical delivery apparatus of claim 6 wherein said first prong comprises an inwardly projecting lip that latches a surface of said carriage after a syringe content administering proximal shifting of said plunger, whereby distal shifting of said plunger after the latching affects a distal shifting of said carriage to retract said carriage and withdraw said needle tip within said housing.

8. The pharmaceutical delivery apparatus of claim 7 wherein said second prong is biased outward by engagement with a projection on said carriage when said first prong latches said carriage, and wherein said housing comprises a lock structured to engage said biased outward second prong when said carriage is retracted to lock said carriage with said needle tip within said housing.

9. A pharmaceutical delivery apparatus comprising:
a housing extending between a distal end and a proximal end;
a syringe carriage rotatably fixed and axially movable within said housing between a first position and second position;
a medication-filled syringe held within said carriage and including a needle having a proximal tip, said needle tip being disposed within said housing when said carriage is in said first position, said needle tip projecting from said housing beyond said proximal end for insertion into an injection site when said carriage is in said second position;
a plunger axially extending from said housing distal end and manually shiftable in the proximal direction to cause needle insertion and injection of medicine from said syringe, said plunger rotatably fixed and axially movable within housing, wherein said plunger includes at least one resilient prong disposed within said housing;
wherein said housing includes at least one camming rib for engagement with said prong;
wherein said carriage includes at least one push surface on an exterior periphery;
wherein said camming rib is structured and arranged to bias said resilient prong inward to engage said at least one push surface during manual proximal plunging of said plunger to affect proximal shifting of said carriage;
a needle cap including a base and a stem, said base exposed at said housing proximal end to be manually grippable for cap removal, said stem upstanding from said base and sized and configured to insert through an opening in said housing proximal end to cover said needle tip when said carriage is in said first position;
means on said carriage and said housing for cooperating with said needle cap to prevent shifting of said plunger in the proximal direction prior to needle cap removal; and
means on said carriage and said housing and said plunger for causing said carriage to retract from said second position and be locked at a position with said needle tip disposed within said housing when the plunged plunger is manually pulled distally from said housing.
